(19) United States
(12) Patent Application Publication
(10) Pub. No.: US 2013/0281663 A1
(43) Pub. Date: Oct. 24, 2013

(54) PREPARATION OF POLYPEPTIDES AND SALTS THEREOF

(71) Applicants: Rama Rao Kvs, Hyderabad (IN); Santhana Krishnan Sriivasan, Tamil Nadu (IN); Basanthi Devi, Hyderabad (IN); Sunil Kumar Gandavadi, Hyderabad (IN); Karthik Ramasamy, Hyderabad (IN); Yagna Kiran Kumar Komaravolu, Hyderabad (IN); Kalyan Chakravarthi Varanasi, Vizianagaram (IN); Ramesh Bocha, Hyderabad (IN); Parameswara Reddy Konche, Kurnool (IN); Laxmi Reddy Katta, Andhra Pradesh (IN); Rajgopal Sharma, Hyderabad (IN); Srilakshmi Nekkalapu, Hyderabad (IN)

(72) Inventors: Rama Rao Kvs, Hyderabad (IN); Santhana Krishnan Sriivasan, Tamil Nadu (IN); Basanthi Devi, Hyderabad (IN); Sunil Kumar Gandavadi, Hyderabad (IN); Karthik Ramasamy, Hyderabad (IN); Yagna Kiran Kumar Komaravolu, Hyderabad (IN); Kalyan Chakravarthi Varanasi, Vizianagaram (IN); Ramesh Bocha, Hyderabad (IN); Parameswara Reddy Konche, Kurnool (IN); Laxmi Reddy Katta, Andhra Pradesh (IN); Rajgopal Sharma, Hyderabad (IN); Srilakshmi Nekkalapu, Hyderabad (IN)

(73) Assignees: DR. REDDY'S LABORATORIES, INC., Bridgewater, NJ (US); DR. REDDY'S LABORATORIES LTD., Hyderabad, Andhra Pradesh (IN)

(21) Appl. No.: 13/639,271

(22) PCT Filed: Apr. 27, 2011
(86) PCT No.: PCT/US11/34102

§ 371 Date: Dec. 13, 2012

Related U.S. Application Data
(60) Provisional application No. 61/356,105, filed on Jun. 18, 2010.
(60) Provisional application No. 61/416,132, filed on Nov. 22, 2010.

(30) Foreign Application Priority Data
Apr. 27, 2010 (IN) 1166/CHE/2010
May 27, 2010 (IN) 1457/CHE/2010
Sep. 27, 2010 (IN) 2845/CHE/2010

(51) Int. Cl. C07K 1/06

(52) U.S. Cl. CPC C07K 1/061
USPC 530/330

(57) ABSTRACT

The application relates to processes for preparing polypeptides. Also provided are processes for preparing glatiramer acetate.

### INTRODUCTION

Aspects of the present application relate to processes for preparing polypeptides. Particular aspects of the present application relate processes for preparing glatiramer acetate.

The drug having the adopted name “glatiramer acetate” (formerly known as copolymer-1) is chemically an acetate salt of a randomized mixture of polymers of L-glutamic acid, L-alanine, L-lysine, and L-tyrosine. It has the structural and chemical formulas of Formula (I).

(Glu, Ala, Lys, Tyr)ₓ.xCH₃COOH (C₅H₉NO₄.C₃H₇NO₂.C₆H₁₄N₂O₂.C₉H₁₁NO₃)ₓ.xC₂H₄O₂ Formula (I)

Glatiramer acetate is the acetate salt of synthetic polypeptides, containing four naturally occurring amino acids: L-glutamic acid, L-alanine, L-tyrosine, and L-lysine with an average molar fraction of 0.141, 0.427, 0.095, and 0.338, respectively. The average molecular weight of glatiramer acetate is 5,000-9,000 Daltons. Glatiramer acetate is the active ingredient in an injectable pharmaceutical product sold by Teva as COPAXONE®, prescribed for reduction of the frequency of relapses in patients with relapsing-remitting multiple sclerosis (RRMS).

U.S. Pat. No. 5,800,808 discloses a process for preparing copolymer-1, by reacting protected copolymer-1 with hydrobromic acid to form trifluoroacetyl copolymer-1, followed by treating the trifluoroacetyl copolymer-1 with aqueous piperidine solution to form copolymer-1 and purifying the resulting copolymer-1.

U.S. Pat. No. 7,495,072 discloses a process for preparing glatiramer acetate, by polymerizing N-carboxyanhydrides of tyrosine, alanine, gamma-benzyl glutamate and N-trifluoroacetyllysine to form protected polypeptides, deprotecting the protected polypeptides with pretreated hydrobromic acid in acetic acid solution to form trifluoroacetyl glatiramer acetate, followed by reacting trifluoroacetyl glatiramer acetate with aqueous piperidine to form a solution of glatiramer acetate and purifying the glatiramer acetate.

The preparation of amino acid N-carboxyanhydrides is discussed in U.S. Pat. No. 7,294,719 B2, involving reacting amino acids, derivatives thereof such as esters, and their salts with carbonylation reagents such as phosgene.

U.S. Pat. No. 7,049,399 discloses a process for the preparation of polypeptide 1, or a pharmaceutically acceptable salt thereof, comprising L-alanine, L-glutamic acid, L-lysine and L-tyrosine randomly arranged in the polypeptide 1 by deprotecting protected copolymer 6 or a salt thereof, to afford polypeptide 1 or a pharmaceutically acceptable salt thereof, in a single step.

U.S. Patent Application Publication No. 2006/0172942 A1 discloses a process for making a mixture of acetate salts of polypeptides, each of which consists of glutamic acid, alanine, tyrosine, and lysine.

U.S. Patent Application Publication No. 2008/0021200 A1 discloses a process for preparing glatiramer acetate by polymerizing a mixture of a N-carboxyanhydride of L-tyrosine, a N-carboxyanhydride of L-alanine, a N-carboxyanhydride of protected L-glutamate, and a N-carboxyanhydride of N-t-butoxycarbonyl-L-lysine, to form a protected glatiramer, followed by treating the protected glatiramer with an acid to form glatiramer.

International Application Publication No. WO 2009/016643 A1 discloses a method of preparation of copolymer-1 fraction (glatiramer acetate, a mixture of polypeptides composed of glutamic acid, alanine, tyrosine, and lysine in a molar ratio of approximately 0.141, 0.427, 0.095, and 0.338) used in pharmaceuticals.

There remains a need for improved processes for the preparation of polypeptides including glatiramer acetate, having high purity, in a cost-effective and environmentally friendly manner.

### SUMMARY

In an aspect, the present application provides processes for preparing polypeptides or pharmaceutically acceptable salts thereof, which include one or more of the following steps, individually or in the sequence recited:

(a) polymerizing a mixture of protected amino acids to form a protected polypeptide;

(b) reacting the protected polypeptide with an acid;

(c) treating the protected polypeptide obtained in step (b) with a reagent; and

(d) reacting the protected polypeptide obtained in step (c) with a base to form a polypeptide or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids selected from L-tyrosine, L-alanine, L-glutamate and L-lysine to form a protected polypeptide;

(b) reacting the protected polypeptide with an acid;

(c) treating the protected polypeptide obtained in step (b) with a reagent; and

(d) reacting the protected polypeptide obtained in step (c) with a base to form a polypeptide or a pharmaceutically acceptable salt thereof.

In an aspect, the present application provides processes for preparing glatiramer or pharmaceutically acceptable salts thereof, which include one or more of the following steps, individually or in the sequence recited:

(a) polymerizing a mixture of protected amino acids consisting of L-tyrosine, L-alanine, L-glutamate and L-lysine to form a protected glatiramer;

(b) reacting the protected glatiramer with an acid;

(c) treating the protected glatiramer obtained in step (b) with a reagent; and

(d) reacting the protected glatiramer obtained in step (c) with a base to form glatiramer or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids to form a protected polypeptide;

(b) reacting the protected polypeptide with a mixture of hydroiodic acid and hypophosphorous acid in acetic acid;

(c) treating the protected polypeptide obtained in step (b) with a reagent; and

(d) reacting the protected polypeptide obtained in step (c) with a base to form a polypeptide or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids selected from L-tyrosine, L-alanine, L-glutamate and L-lysine to form a protected polypeptide;

(b) reacting the protected polypeptide with a mixture of hydroiodic acid and hypophosphorous acid in acetic acid;

(c) treating the protected polypeptide obtained in step (b) with a reagent; and

(d) reacting the protected polypeptide obtained in step (c) with a base to form a polypeptide or a pharmaceutically acceptable salt thereof.

In an aspect, the present application provides processes for preparing glatiramer or pharmaceutically acceptable salts thereof, which include one or more of the following steps, individually or in the sequence recited:

(a) polymerizing a mixture of protected amino acids consisting of L-tyrosine, L-alanine, L-glutamate, and L-lysine to form a protected glatiramer;

(b) reacting the protected glatiramer with a mixture of hydroiodic acid and hypophosphorous acid in acetic acid;

(c) treating the protected glatiramer obtained in step (b) with a reagent; and

(d) reacting the protected glatiramer obtained in step (c) with a base to form glatiramer or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids to form a protected polypeptide;

(b) reacting the protected polypeptide with an acid comprising hydroiodic acid and hypophosphorous acid;

(c) treating the protected polypeptide obtained in step (b) with a reagent; and

(d) reacting the protected polypeptide obtained in step (c) with a base to form a polypeptide or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids selected from L-tyrosine, L-alanine, L-glutamate and L-lysine to form a protected polypeptide;

(b) reacting the protected polypeptide with an acid comprising hydroiodic acid and hypophosphorous acid;

(c) treating the protected polypeptide obtained in step (b) with a reagent; and

(d) reacting the protected polypeptide obtained in step (c) with a base to form a polypeptide or a pharmaceutically acceptable salt thereof.

In an aspect, the present application provides processes for preparing glatiramer or pharmaceutically acceptable salts thereof, which includes one or more of the following steps, individually or in the sequence recited:

(a) polymerizing a mixture of protected amino acids consisting of L-tyrosine, L-alanine, L-glutamate and L-lysine to form a protected glatiramer;

(b) reacting the protected glatiramer with an acid comprising hydroiodic acid and hypophosphorous acid;

(c) treating the protected glatiramer obtained in step (b) with a reagent; and

(d) reacting the protected glatiramer obtained in step (c) with a base to form glatiramer or a pharmaceutically acceptable salt thereof.

In an aspect, the present application provides processes for preparing polypeptide or pharmaceutically acceptable salts thereof, which include one or more of the following steps, individually or in the sequence recited:

(a) polymerizing a mixture of protected amino acids to form a protected polypeptide;

(b) reacting the protected polypeptide with an acid comprising hydroiodic acid;

(c) treating the protected polypeptide obtained in step (b) with a reagent; and

(d) reacting the protected polypeptide obtained in step (c) with a base to form a polypeptide or a pharmaceutically acceptable salt thereof.

In an aspect, the present application provides a process for preparing polypeptides or pharmaceutically acceptable salts thereof, which include one or more of the following steps, individually or in the sequence recited:

(a) polymerizing a mixture of protected amino acids selected from L-tyrosine, L-alanine, L-glutamate and L-lysine to form a protected polypeptide;

(b) reacting the protected polypeptide with an acid comprising hydroiodic acid;

(c) treating the protected polypeptide obtained in step (b) with a reagent; and

(d) reacting the protected polypeptide obtained in step (c) with a base to form a polypeptide or a pharmaceutically acceptable salt thereof.

In an aspect, the present application provides processes for preparing glatiramer or pharmaceutically acceptable salts thereof, which include one or more of the following steps, individually or in the sequence recited:

(a) polymerizing a mixture of protected amino acids consisting of L-tyrosine, L-alanine, L-glutamate and L-lysine to form a protected glatiramer;

(b) reacting the protected glatiramer with an acid comprising hydroiodic acid;

(c) treating the protected glatiramer obtained in step (b) with a reagent; and

(d) reacting the protected glatiramer obtained in step (c) with a base to form glatiramer or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids to form a protected polypeptide;

(b) reacting the protected polypeptide with an acid comprising hydrochloric acid;

(c) treating the protected polypeptide obtained in step (b) with a reagent; and

(d) reacting the protected polypeptide obtained in step (c) with a base to form a polypeptide or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids selected from L-tyrosine, L-alanine, L-glutamate and L-lysine to form a protected polypeptide;

(b) reacting the protected polypeptide with an acid comprising hydrochloric acid;

(c) treating the protected polypeptide obtained in step (b) with a reagent; and

(d) reacting the protected polypeptide obtained in step (c) with a base to form a polypeptide or a pharmaceutically acceptable salt thereof.

In an aspect, the present application provides processes for preparing glatiramer or pharmaceutically acceptable salts thereof, which include one or more of the following steps, individually or in the sequence recited:

(a) polymerizing a mixture of protected amino acids consisting of L-tyrosine, L-alanine, L-glutamate and L-lysine to form a protected glatiramer;

(b) reacting the protected glatiramer with an acid comprising hydrochloric acid;

(c) treating the protected glatiramer obtained in step (b) with a reagent; and

(d) reacting the protected glatiramer obtained in step (c) with a base to form glatiramer or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids to form a protected polypeptide;

(b) reacting the protected polypeptide with a solution of hydrobromic acid in acetic acid;

(c) treating the protected polypeptide obtained in step (b) with a reagent; and

(d) reacting the protected polypeptide obtained in step (c) with a base to form a polypeptide or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids selected from L-tyrosine, L-alanine, L-glutamate and L-lysine to form a protected polypeptide;

(b) reacting the protected polypeptide with a solution of hydrobromic acid in acetic acid;

(c) treating the protected polypeptide obtained in step (b) with a reagent; and

(d) reacting the protected polypeptide obtained in step (c) with a base to form a polypeptide or a pharmaceutically acceptable salt thereof.

In an aspect, the present application provides processes for preparing glatiramer or pharmaceutically acceptable salts thereof, which include one or more of the following steps, individually or in the sequence recited:

(a) polymerizing a mixture of protected amino acids consisting of L-tyrosine, L-alanine, L-glutamate and L-lysine to form a protected glatiramer;

(b) reacting the protected glatiramer with a solution of hydrobromic acid in acetic acid;

(c) treating the protected glatiramer obtained in step (b) with a reagent; and

(d) reacting the protected glatiramer obtained in step (c) with a base to form glatiramer or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids to form a protected polypeptide;

(b) reacting the protected polypeptide with a mixture of hydroiodic acid and hypophosphorous acid in acetic acid; and

(c) reacting the protected polypeptide obtained in step (b) with a base to form a polypeptide or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids selected from L-tyrosine, L-alanine, L-glutamate and L-lysine to form a protected polypeptide;

(b) reacting the protected polypeptide with a mixture of hydroiodic acid and hypophosphorous acid in acetic acid; and

(c) reacting the protected polypeptide obtained in step (b) with a base to form a polypeptide or a pharmaceutically acceptable salt thereof.

In an aspect, the present application provides processes for preparing glatiramer or pharmaceutically acceptable salts thereof, which includes one or more of the following steps, individually or in the sequence recited:

(a) polymerizing a mixture of protected amino acids consisting of L-tyrosine, L-alanine, L-glutamate and L-lysine to form a protected glatiramer;

(b) reacting the protected glatiramer with a mixture of hydroiodic acid and hypophosphorous acid in acetic acid; and

(c) reacting the protected glatiramer obtained in step (b) with a base to form glatiramer or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids to form a protected polypeptide;

(b) reacting the protected polypeptide with an acid comprising hydroiodic acid and hypophosphorous acid; and

(c) reacting the protected polypeptide obtained in step (b) with a base to form a polypeptide or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids selected from L-tyrosine, L-alanine, L-glutamate and L-lysine to form a protected polypeptide;

(b) reacting the protected polypeptide with an acid comprising hydroiodic acid and hypophosphorous acid; and

(c) reacting the protected polypeptide obtained in step (b) with a base to form a polypeptide or a pharmaceutically acceptable salt thereof.

In an aspect, the present application provides process for preparing glatiramer or pharmaceutically acceptable salts thereof, which include one or more of the following steps, individually or in the sequence recited:

(a) polymerizing a mixture of protected amino acids consisting of L-tyrosine, L-alanine, L-glutamate and L-lysine to form a protected glatiramer;

(b) reacting the protected glatiramer with an acid comprising hydroiodic acid and hypophosphorous acid; and

(c) reacting the protected glatiramer obtained in step (b) with a base to form glatiramer or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids to form a protected polypeptide;

(b) reacting the protected polypeptide with an acid comprising hydroiodic acid; and

(c) reacting the protected polypeptide obtained in step (b) with a base to form a polypeptide or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids selected from L-tyrosine, L-alanine, L-glutamate and L-lysine to form a protected polypeptide;

(b) reacting the protected polypeptide with an acid comprising hydroiodic acid; and

(c) reacting the protected polypeptide obtained in step (b) with a base to form a polypeptide or a pharmaceutically acceptable salt thereof.

In an aspect, the present application provides processes for preparing glatiramer or pharmaceutically acceptable salts thereof, which include one or more of the following steps, individually or in the sequence recited:

(a) polymerizing a mixture of protected amino acids consisting of L-tyrosine, L-alanine, L-glutamate and L-lysine to form a protected glatiramer;

(b) reacting the protected glatiramer with an acid comprising hydroiodic acid; and

(c) reacting the protected glatiramer obtained in step (b) with a base to form glatiramer or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids to form a protected polypeptide;

(b) reacting the protected polypeptide with an acid comprising hydrochloric acid; and

(c) reacting the protected polypeptide obtained in step (b) with piperidine to form a polypeptide or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids selected from L-tyrosine, L-alanine, L-glutamate and L-lysine to form a protected polypeptide;

(b) reacting the protected polypeptide with an acid comprising hydrochloric acid; and

(c) reacting the protected polypeptide obtained in step (b) with piperidine to form a polypeptide or a pharmaceutically acceptable salt thereof.

In an aspect, the present application provides processes for preparing glatiramer or pharmaceutically acceptable salts thereof, which include one or more of the following steps, individually or in the sequence recited:

(a) polymerizing a mixture of protected amino acids consisting of L-tyrosine, L-alanine, L-glutamate and L-lysine to form a protected glatiramer;

(b) reacting the protected glatiramer with an acid comprising hydrochloric acid;

(c) reacting the protected glatiramer obtained in step (b) with piperidine to form glatiramer or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids to form a protected polypeptide;

(b) reacting the protected polypeptide with an acid comprising sulphuric acid; and

(c) reacting the protected polypeptide obtained in step (b) with piperidine to form a polypeptide or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids selected from L-tyrosine, L-alanine, L-glutamate and L-lysine to form a protected polypeptide;

(b) reacting the protected polypeptide with an acid comprising sulphuric acid; and

(c) reacting the protected polypeptide obtained in step (b) with piperidine to form a polypeptide or a pharmaceutically acceptable salt thereof.

In an aspect, the present application provides processes for preparing glatiramer or pharmaceutically acceptable salts thereof, which include one or more of the following steps, individually or in the sequence recited:

(a) polymerizing a mixture of protected amino acids consisting of L-tyrosine, L-alanine, L-glutamate and L-lysine to form a protected glatiramer;

(b) reacting the protected glatiramer with an acid comprising sulphuric acid; and

(c) reacting the protected glatiramer obtained in step (b) with piperidine to form glatiramer or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids to form a protected polypeptide; and

(b) reacting the protected polypeptide with a mixture of hydroiodic acid and hypophosphorous acid in acetic acid to form a polypeptide or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids selected from L-tyrosine, L-alanine, L-glutamate and L-lysine to form a protected polypeptide; and

(b) reacting the protected polypeptide with a solution of hydrooiodic acid and hypophosphorous acid in acetic acid to form a polypeptide or a pharmaceutically acceptable salt thereof.

In an aspect, the present application provides processes for preparing glatiramer or pharmaceutically acceptable salts thereof, which include one or more of the following steps, individually or in the sequence recited:

(a) polymerizing a mixture of protected amino acids consisting of L-tyrosine, L-alanine, L-glutamate and L-lysine to form a protected glatiramer; and

(b) reacting the protected glatiramer with a mixture of hydroiodic acid and hypophosphorous acid in acetic acid to form glatiramer or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids to form a protected polypeptide;

(b) reacting the protected polypeptide with an acid comprising hydroiodic acid and hypophosphorous acid to form a polypeptide or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids selected form L-tyrosine, L-alanine, L-glutamate and L-lysine to form a protected polypeptide; and

(b) reacting the protected polypeptide with an acid comprising hydrooiodic acid and hypophosphorous acid to form a polypeptide or a pharmaceutically acceptable salt thereof.

In an aspect, the present application provides process for preparing glatiramer or pharmaceutically acceptable salts thereof, which include one or more of the following steps, individually or in the sequence recited:

(a) polymerizing a mixture of protected amino acids consisting of L-tyrosine, L-alanine, L-glutamate and L-lysine to form a protected glatiramer; and

(b) reacting the protected glatiramer with an acid comprising hydroiodic acid and hypophosphorous acid to form glatiramer or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected amino acids to form a protected polypeptide; and

(b) reacting the protected polypeptide with an acid comprising hydroiodic acid to form a polypeptide or a pharmaceutically acceptable salt thereof.

In an aspect, the present application provides processes for preparing polypeptides or pharmaceutically acceptable salts thereof, which include one or more of the following steps, individually or in the sequence recited:

(a) polymerizing a mixture of protected selected from L-tyrosine, L-alanine, L-glutamate and L-lysine to form a protected polypeptide; and

(b) reacting the protected polypeptide with an acid comprising hydrooiodic acid to form a polypeptide or a pharmaceutically acceptable salt thereof.

In an aspect, the present application provides processes for preparing glatiramer or pharmaceutically acceptable salts thereof, which include one or more of the following steps, individually or in the sequence recited:

(a) polymerizing a mixture of protected amino acids consisting of L-tyrosine, L-alanine, L-glutamate and L-lysine to form a protected glatiramer; and

(b) reacting the protected glatiramer with an acid comprising hydroiodic acid to form glatiramer or a pharmaceutically acceptable salt thereof.

### DETAILED DESCRIPTION

In aspects of the present application, processes for preparing polypeptides or pharmaceutically acceptable salts thereof include a step of polymerizing a mixture of protected amino acids to form a protected polypeptide.

Polymerizing a mixture of protected amino acids to form a protected polypeptide may be carried out in the presence of one or more suitable initiators. Suitable initiators that may be used in polymerization reactions include, but are not limited to, alkyl amines, such as, for example, dimethylamine, diethylamine, di-n-propylamine, diisopropylamine, triethylamine, N-ethylmethylamine, di-n-butylamine, diisobutylamine, di-sec-butylamine, di-tert-butylamine, diamylamine, di-n-octylamine, di-(2-ethylhexyl)amine, di-iso-nonylamine, diallylamine, N-methylaniline, diphenylamine, hexylamine, phenethylamine, and the like. Other useful initiators include aziridine, pyrrole, pyrrolidine, imidazole, indole, piperidine, purine, sodium methoxide, potassium t-butoxide, sodium hydride, potassium hydride, 2,2,6,6-tetramethylpiperidine, dicyclohexylamine, dicyclohexylundecane (DCU), lithium diisopropylamide, t-butyllithium, and the like; ion exchange resins including resins bound to ions, such as, for example, sodium, potassium, lithium, calcium, magnesium, substituted or unsubstituted ammonium, and the like. Combinations of any two or more initiators also are useful.

The quantities of initiator that may be used in polymerization reactions may be less than about 5%, less than about 4%, less than about 3%, less than about 2%, less than about 1%, less than about 0.5%, less than about 0.25%, less than about 0.1%, less than about 0.05%, less than about 0.01%, and any other suitable quantities, based on the weight of the mixture of protected amino acids.

Polymerization of protected amino acids to form protected polypeptides may be conducted in a solvent. Suitable solvents that may be used include, but are not limited to: ethers, such as, for example, diethyl ether, diisopropyl ether, tert-butyl methyl ether, dibutyl ether, tetrahydrofuran, dimethylfuran, 1,2-dimethoxyethane, 2-methoxyethanol, 2-ethoxyethanol, anisole, 1,4-dioxane, and the like; esters, such as, for example, ethyl formate, methyl acetate, ethyl acetate, propyl acetate, butyl acetate, methyl propanoate, ethyl propanoate, methyl butanoate, ethyl butanoate, and the like; aliphatic or alicyclic hydrocarbons, such as, for example, hexane, heptane, pentane, cyclohexane, methylcyclohexane, and the like; nitromethane; halogenated hydrocarbons, such as, for example, dichloromethane, chloroform, 1,1,2-trichloroethane, 1,2-dichloroethene, and the like; aromatic hydrocarbons, such as, for example, toluene, xylene, chlorobenzene, tetraline, and the like; nitriles, such as, for example, acetonitrile, propionitrile, and the like; polar aprotic solvents, such as, for example, N,N-dimethylformamide, N,N-dimethylacetamide, N-methylpyrrolidone, pyridine, dimethylsulfoxide, sulfolane, formamide, acetamide, propanamide, and the like; including any mixtures of two or more thereof.

Suitable temperatures for the polymerization reactions may be less than about 55° C., less than about 45° C., less than about 35° C., less than about 25° C., less than about 15° C., less than about 10° C., or any other suitable temperatures.

Separation of protected polypeptide may be accomplished by combining the reaction mixture with water, which results in precipitation of the protected polypeptide. Suitable temperatures for separation of protected polypeptide may be less than about 50° C., less than about 40° C., less than about 30° C., less than about 20° C., less than about 10° C., or any other suitable temperatures. Suitable times for separation may be less than about 5 hours, less than about 3 hours, less than about 2 hours, less than about 1 hour, less than about 45 minutes, or any longer times. The exact temperatures and times required for complete separation may be readily determined by a person skilled in the art and will also depend on parameters, such as, for example, concentration and temperature of the solution or slurry. Stirring or other alternate methods, such as, for example, shaking, agitation, or the like, that mix the contents may also be employed for separation.

The separated protected polypeptide may be recovered by methods including decantation, centrifugation, gravity filtration, suction filtration, or any other techniques for the recovery of solids.

The recovered protected polypeptide may be optionally dried. Drying may be carried out in a tray dryer, vacuum oven, air oven, fluidized bed dryer, spin flash dryer, flash dryer, and the like. The drying may be carried out at atmospheric pressure or under a reduced pressure, at temperatures less than about 55° C., less than about 45° C., less than about 35° C., less than about 25° C., or any other suitable temperatures. For example, drying times may vary from about 1 to about 10 hours, or longer.

Aspects of the present application include a step of reacting a protected polypeptide with an acid.

Suitable acids that may be used in the reaction of the protected polypeptide with one or more suitable acids, include, but are not limited to, acetic acid, propionic acid, butyric acid, hydrochloric acid, hydrogen bromide, hydrogen fluoride, hydrogen iodide (hydroiodic acid), methanesulfonic acid, trifluoromethanesulfonic acid, phosphorous acid, trifluoroacetic acid, sulfuric acid, phosphoric acid and hypo phosphoric acid; or the like; or mixtures thereof. The quantities of acid that may be used in the reaction of the protected polypeptide with one or more suitable acids may be less than about 50 times, less than about 40 times, less about 30 times, less than about 20 times, less than about 10 times, less than about 5 times, by volume, the weight of protected polypeptide. Suitably the said acid may have a concentration of not less than about 30% by weight. For varying concentrations of the acid, the quantity of acid to be used in the reaction of the protected polypeptide with one or more suitable acids may be readily calculated by one skilled in the art.

In embodiments, the acid that is employed may cleave protecting groups from the protected polypeptide to form a polypeptide, or form a pharmaceutically acceptable salt thereof.

Suitable temperatures that may be used in the reaction of the protected polypeptide with one or more suitable acids may be less than about 60° C., less than about 50° C., less than about 40° C., less than about 30° C., less than about 25° C., less than about 15° C., less than about 10° C., less than about 5° C., less than about 0° C., or any other suitable temperatures.

Suitable solvents that may be used in the reaction of the protected polypeptide with one or more suitable acids include, but are not limited to: ethers, such as, for example, diethyl ether, diisopropyl ether, tert-butyl methyl ether, dibutyl ether, tetrahydrofuran, 1,2-dimethoxyethane, 2-methoxyethanol, 2-ethoxyethanol, anisole, 1,4-dioxane, and the like; esters, such as, for example, ethyl formate, methyl acetate, ethyl acetate, propyl acetate, butyl acetate, methyl propanoate, ethyl propanoate, methyl butanoate, ethyl butanoate, and the like; aliphatic or alicyclic hydrocarbons, such as, for example, hexane, heptane, pentane, cyclohexane, methylcyclohexane, and the like; nitromethane; halogenated hydrocarbons, such as, for example, dichloromethane, chloroform, 1,1,2-trichloroethane, 1,2-dichloroethene, and the like; aromatic hydrocarbons, such as, for example, toluene, xylene, chlorobenzene, tetralin, and the like; nitriles, such as, for example, acetonitrile, propionitrile, and the like; polar aprotic solvents, such as, for example, N,N-dimethylformamide, N,N-dimethylacetamide, N-methylpyrrolidone, pyridine, dimethylsulfoxide, sulfolane, formamide, acetamide, propanamide, and the like; acetic acid, and the like; and any mixtures of two or more thereof.

The separation of protected polypeptide or protected glatiramer may be accomplished by methods including removal of solvent, cooling, concentrating the reaction mass, combining with an anti-solvent, and the like. In embodiments, the separation of protected polypeptide may be effected by addition of the reaction mixture to water, which results in precipitation of the protected polypeptide or protected glatiramer. Suitable temperatures for separation may be less than about 50° C., less than about 40° C., less than about 30° C., less than about 20° C., less than about 10° C., or any other suitable temperatures. Suitable times for separation may be less than about 5 hours, less than about 3 hours, less than about 2 hours, less than about 1 hour, less than about 45 minutes. The exact temperatures and times required for complete separation may be readily determined by a person skilled in the art and will also depend on parameters, such as, for example, concentration and temperature of the solution or slurry. Stirring or other alternate methods, such as, for example, shaking, agitation, or the like, that mix the contents may also be employed for separation.

The separated protected polypeptide or protected glatiramer may be recovered by methods including decantation, centrifugation, gravity filtration, suction filtration, or any other techniques for the recovery of solids.

The recovered solid may optionally be dried. Drying may be carried out in a tray dryer, vacuum oven, air oven, fluidized bed dryer, spin flash dryer, flash dryer, or the like. The drying may be carried out at atmospheric pressure or under a reduced pressure, at temperatures less than about 55° C., or less than about 45° C., or less than about 35° C., or less than about 25° C., or any other suitable temperatures. In embodiments, drying times may vary from about 1 to about 10 hours, or longer.

Aspects of the present application include a step of treating the protected polypeptide or protected glatiramer, obtained by reacting the protected polypeptide with an acid, with a reagent, prior to use in the reaction of protected polypeptide or protected glatiramer with a base to form a polypeptide or glatiramer.

Treating the protected polypeptide or protected glatiramer with a reagent may be effected by methods including washing, slurrying, quenching, and the like.

The content of molecular species in acid or acid combinations that may be used in the reaction of the protected polypeptide with an acid, may have an important role in the formation of functionalized polypeptides in polypeptides or glatiramer.

For example, the content of molecular halogen or free halogen species in acids or acid combinations that may be used in the reaction of the protected polypeptide with an acid, may play an important role in the formation of halogenated polypeptides in polypeptides or glatiramer.

It has been discovered that protected polypeptide or protected glatiramer, containing molecular species originating from acids or acid combinations that are used for preparing it, may involve functional transformation with one or more functional groups of polypeptides while reacting the protected polypeptide or protected glatiramer with a base to form a polypeptide or glatiramer, and result in the functionalized polypeptide or functionalized glatiramer being present as a contaminant in the obtained polypeptide or glatiramer.

For example, protected polypeptide or protected glatiramer, containing molecular halogen or free halogen species bound to the surface, may interact with one or more functional groups of polypeptides while reacting the protected polypeptide or protected glatiramer with a base to form a polypeptide or glatiramer, and result in the halogenated polypeptide or halogenated glatiramer being present as a contaminant in the obtained polypeptide or glatiramer.

This can be prevented by treating the protected polypeptide or protected glatiramer, obtained by the reaction of protected polypeptide with an acid, with a reagent prior to use in the reaction of protected polypeptide or protected glatiramer with a base, resulting in the formation of protected polypeptide or protected glatiramer that is substantially free of molecular species.

For instance, treatment of the protected polypeptide or protected glatiramer, obtained by the reaction of protected polypeptide with an acid, with a reagent prior to use in the reaction of protected polypeptide or protected glatiramer with a base, may lead to the formation of protected polypeptide or protected glatiramer substantially free of molecular halogen or free halogen species.

Suitable reagents that may be used for this treatment to reduce the content of molecular impurities include, but are not limited to: alkali or alkaline earth metal thiosulfates, such as, for example, sodium thiosulfate and the like; alkali metal bisulfates, such as, for example, sodium bisulfate and the like; alkali metal metabisulfites, such as, for example, sodium metabisulfite and the like; ascorbic acid; activated carbon fibers; solutions of an organic-soluble ion exchange resin, for example, Amberlite® LA-2 and the like; silver salts; sodium bicarbonate; and the like.

Amberlite LA-2 is liquid highly-branched secondary amines, having molecular weights averaging about 350-400, binding capacity about 2.2-2.3 meq/mL, and the CAS No. 11128-96-4. It is soluble in organic solvents and insoluble in aqueous media.

The protected polypeptide or protected glatiramer, obtained by treating the protected polypeptide or protected glatiramer with a reagent, may be further washed with a solvent. Suitable solvents that may be used include, but are not limited to: water, aliphatic or alicyclic hydrocarbons, such as, for example, hexane, heptane, pentane, cyclohexane, methylcyclohexane, and the like; ethers, such as, for example, diethyl ether, diisopropyl ether, tert-butyl methyl ether, dibutyl ether, tetrahydrofuran, 1,2-dimethoxyethane, 2-methoxyethanol, 2-ethoxyethanol, anisole, 1,4-dioxane, and the like; esters, such as, for example, ethyl formate, methyl acetate, ethyl acetate, propyl acetate, butyl acetate, methyl propanoate, ethyl propanoate, methyl butanoate, ethyl butanoate, and the like; and any mixtures of two or more thereof.

In embodiments, protected polypeptides or protected glatiramer, prepared according to a process described in the present application, have peak average molecular weights ranging from about 2000 Daltons to about 40,000 Daltons, or from about 4000 Daltons to about 18,000 Daltons, or about 4000 Daltons to about 13,000 Daltons, or from about 5000 Daltons to about 9000 Daltons, as determined using techniques such as gel permeation chromatography (GPC).

Aspects of the present application include a step of reacting the protected polypeptide or protected glatiramer with a base.

Bases that may be used in the reaction of protected polypeptide or protected glatiramer with a base to form a polypeptide or protected glatiramer, or a pharmaceutically acceptable salt thereof, include, but are not limited to: organic bases, such as, for example, triethylamine, tributylamine, N-methylmorpholine, N,N-diisopropylethylamine, N-methylpyrrolidine, piperidine, aqueous piperidine, pyrrolidine pyridine, 4-(N,N-dimethylamino)pyridine, morpholine, imidazole, 2-methylimidazole, 4-methylimidazole, methanolic ammonia, and the like; inorganic bases, including: alkali metal hydroxides, such as, for example, lithium hydroxide, sodium hydroxide, potassium hydroxide, and cesium hydroxide; alkaline earth metal hydroxides, such as, for example, barium hydroxide, magnesium hydroxide, calcium hydroxide, and the like; alkali metal carbonates, such as, for example, sodium carbonate, potassium carbonate, lithium carbonate, cesium carbonate, and the like, alkaline earth metal carbonates, such as, for example, magnesium carbonate, calcium carbonate, barium carbonate, and the like; alkali metal bicarbonates, such as, for example, lithium bicarbonate, sodium bicarbonate, potassium bicarbonate, and the like; and mixtures of any two or more thereof.

The reaction of protected polypeptide or protected glatiramer with a base to form a polypeptide or glatiramer or a pharmaceutically acceptable salt thereof may be carried out in a solvent. Suitable solvents that may be used in the reaction of protected polypeptide with a base to form a polypeptide or glatiramer include, but are not limited to: water, ethers, such as, for example, diethyl ether, diisopropyl ether, tert-butyl methyl ether, dibutyl ether, tetrahydrofuran, 1,2-dimethoxyethane, 2-methoxyethanol, 2-ethoxyethanol, anisole, 1,4-dioxane, and the like; esters, such as, for example, ethyl formate, methyl acetate, ethyl acetate, propyl acetate, butyl acetate, methyl propanoate, ethyl propanoate, methyl butanoate, ethyl butanoate, and the like; aliphatic or alicyclic hydrocarbons, such as, for example, hexane, heptane, pentane, cyclohexane, methylcyclohexane, and the like; nitromethane; halogenated hydrocarbons, such as, for example, dichloromethane, chloroform, 1,1,2-trichloroethane, 1,2-dichloroethene, and the like; aromatic hydrocarbons, such as, for example, toluene, xylene, chlorobenzene, tetralin, and the like; nitriles, such as, for example, acetonitrile, propionitrile, and the like; polar aprotic solvents, such as, for example, N,N-dimethylformamide, N,N-dimethylacetamide, N-methylpyrrolidone, pyridine, dimethylsulfoxide, sulfolane, formamide, acetamide, propanamide, and the like; acetic acid and the like; and any mixtures of two or more thereof.

Suitable temperatures that may be used in the reaction of protected polypeptide with a base to form a polypeptide or glatiramer are less than about 60° C., less than about 55° C., less than about 50° C., less than about 45° C., less than about 40° C., less than about 35° C., less than about 30° C., less than about 25° C., less than about 15° C., less than about 10° C., less than about 5° C., less than about 0° C., or any other suitable temperatures.

In an aspect, the polypeptide or glatiramer prepared according to the processes of the present application may be purified. Purification may be performed using any techniques, including methods that are known in the art. In embodiments, purification of polypeptide or glatiramer may use methods such as dialysis or ultrafiltration.

In embodiments, the polypeptide or glatiramer is subjected to diafiltration against water or buffering agents, such as acetate buffers, phosphate buffers, or citrate buffers, using a molecular weight cutoff membrane (e.g., 1 KDa, 2 KDa, 3 KDa, and 30 KDa) in step or constant modes of operation. In embodiments, diafiltration solutions can be acidified with a weak acid, such as aqueous acetic acid, and dialyzed against water. For example, concentrations of acetic acid may be less than about 1%, or less than about 0.5%, by volume.

The final dialyzed solution obtained by concentration through an ultrafiltration membrane can be lyophilized to form substantially pure polypeptide or substantially pure glatiramer, or pharmaceutically acceptable salts thereof.

The phrase, “substantially pure,” as used herein above, unless otherwise defined, refers to polypeptide, glatiramer, or pharmaceutically acceptable salts thereof that is substantially free of one or more polypeptide fragments having molecular weights higher than about 40 KDa, or substantially free of polypeptide fragments having molecular weights less than about 2 KDa.

The phrase, “substantially free,” as used herein above, unless otherwise defined, refers to polypeptide, glatiramer, or pharmaceutically acceptable salts thereof containing less than about 5%, less than about 3%, less than about 2%, less than about 1%, or less than about 0.5%, by weight, of one or more of the corresponding species of polypeptides having a molecular weight of about 40 KDa or higher, or polypeptide fragments having a molecular weight of about 2 KDa or less.

In embodiments, polypeptides, or pharmaceutically acceptable salts thereof, prepared according to a process described in the present application may have peak average molecular weights ranging from about 2,000 Daltons to about 40,000 Daltons, or from about 4,000 Daltons to about 18,000 Daltons, or from about 4,000 Daltons to about 13,000 Daltons, or from about 5,000 Daltons to about 9,000 Daltons, as determined using techniques such as gel permeation chromatography (GPC).

In embodiments, glatiramer, or pharmaceutically acceptable salts thereof, prepared according to a process described in the present application may have peak average molecular weights ranging from about 5,000 Daltons to about 9,000 Daltons, as determined using techniques such as gel permeation chromatography (GPC).

In embodiments, polypeptides, or pharmaceutically acceptable salts thereof, prepared according to a process described in the present application have at least 75% of their molar fraction within the molecular weight range of about 2,000 Daltons to about 20,000 Daltons.

In embodiments, glatiramer acetate prepared according to a process described in the present application has at least 75% of its molar fraction within the molecular weight range of about 2,000 Daltons to about 20,000 Daltons.

A gel permeation chromatography method that is useful for determining the molecular weights of polypeptides or pharmaceutically acceptable salts thereof utilizes a Superose™ 12, 10×300-310 mm, 11 μm, or equivalent column. Additional parameters are as shown in Table 1.

TABLE 1

| | |
|---|---|
| Flow rate | 0.5 mL/minute (isocratic). |
| Detector | 210 nm. |
| Column temperature | Less than 30° C. |
| Concentration | 4 mg/mL. |
| Mobile phase | Buffer: Na₂HPO₄ and NaCl solution |
| Injection volume | 50 μL. |
| Run time | 60 minutes for standard and 90 minutes |
| | for sample. |

The molar fractions of the amino acids in the polypeptide may be determined using methods known in the art. For example, a sample solution is prepared using 2 mg of the polypeptide and hydrolyzed using 6N HCl, under a N₂ atmosphere at about 110-130° C. Amino acid standard solutions containing each of glutamic acid, alanine, tyrosine, and lysine hydrochloride are prepared. The standard and sample solutions are derivatized with fluorenylmethyloxycarbonyl (Fmoc) reagent. The standard and sample solutions can be analyzed using a C18 or equivalent column, in an instrument equipped with a UV detector. Additional parameters are as shown in Table 2.

TABLE 2

| | |
|---|---|
| Flow rate | 1.0 mL/minute. |
| Detector | 265 nm. |
| Column temperature | 30° C. |
| Mobile phases | Mobile phase A: Mix a pH 3.5 buffer (sodium |
| | acetate trihydrate and acetic acid) and |
| | acetonitrile in the volume ratio 90:10. |
| | Mobile phase B: Mix a pH 3.5 buffer (sodium |
| | acetate trihydrate and acetic acid) and |
| | acetonitrile in the volume ratio 10:90. |
| Injection volume | 50 μL. |
| Elution | Gradient. |

The molar fractions of the amino acids in the polypeptide sample are determined based on peak areas.

Protected polypeptides obtained according to a process of the present application may be substantially free of benzyl chloride.

Protected glatiramer obtained according to a process of the present application may be substantially free of benzyl chloride.

Trifluoroacetyl glatiramer obtained according to a process of the present application may be substantially free of benzyl chloride.

Polypeptides obtained according to a process of the present application may be substantially free of benzyl chloride.

Glatiramer acetate obtained according to a process of the present application may be substantially free of benzyl chloride.

The phrase, “substantially free,” in this context, means that the compound contains less than about 3%, less than about 2%, less than about 1%, less than about 0.5%, less than about 0.3%, less than about 0.1%, less than about 0.05%, or less than about 0.01%, by weight of benzyl chloride, as determined using high performance liquid chromatography (HPLC).

A HPLC method for the analysis of the benzyl chloride content utilizes a C18 or equivalent column. Additional parameters are as shown in Table 3.

TABLE 3

| | |
|---|---|
| Flow rate | 1.0 mL/minute. |
| Column temperature | Ambient. |
| Mobile phases | Mobile phase A: 0.1% OPA in water and |
| | acetonitrile (90:10 by volume). |
| | Mobile phase B: 0.1% OPA in water and |
| | acetonitrile (10:90 by volume). |
| | OPA: Orthophosphoric acid. |
| Injection volume | 10 μL. |
| Elution | Gradient. |

Polypeptides or pharmaceutically acceptable salts thereof prepared according to a process of the present application may be substantially free of one or more of its corresponding functionalized polypeptides, e.g., the polypeptides, wherein the one or more functional groups are mono-, di- or poly-functionalized, as determined by HPLC.

For example, polypeptides or pharmaceutically acceptable salts thereof prepared according to a process of the present application may be substantially free of one or more of its corresponding halogenated polypeptides, e.g., polypeptides wherein the tyrosine moiety is mono-, di-, or poly-halogenated. Examples of halogens are chlorine, bromine, and iodine.

Glatiramer acetate obtained according to a process of the present application may be substantially free of one or more of its corresponding halogenated polypeptides, e.g., polypeptides wherein the tyrosine moiety is mono-, di-, or poly-halogenated. Examples of halogens are chlorine, bromine, and iodine.

The phrase, “substantially free” of functionalized polypeptides, as used herein, means less than about 2%, less than about 1%, less than about 0.5%, less than about 0.3%, less than about 0.1%, less than about 0.05%, less than about 0.01%, less than about 0.005%, or less than about 0.001%, by weight, as determined using techniques such as HPLC. Functionalized polypeptides, as used herein, unless otherwise defined refer to the polypeptides, wherein the one or more functional groups are mono-, di-, or poly-functionalized.

The phrase, “substantially free” of halogenated polypeptides, as used herein, means less than about 2%, less than about 1%, less than about 0.5%, less than about 0.3%, less than about 0.1%, less than about 0.05%, less than about 0.01%, less than about 0.005%, or less than about 0.001%, by weight, as determined using HPLC. Halogenated polypeptides, as used herein, unless otherwise defined refer to the polypeptides, wherein the tyrosine moiety is mono-, di-, or poly-halogenated. Examples of halogens are chlorine, bromine, and iodine.

All percentages and ratios used herein are by weight of the total composition and all measurements made are at 25° C. and atmospheric pressure, unless otherwise designated. All temperatures are in degrees Celsius unless specified otherwise. As used herein, “comprising” means the elements recited, or their equivalent in structure or function, plus any other element or elements that are not recited. The terms “containing,” “having,” and “including” are also to be construed as open ended unless the context suggests otherwise. As used herein, “consisting essentially of” means that the application may include ingredients in addition to those recited in the claim, but only if the additional ingredients do not materially alter the basic and novel characteristics of the claimed application. All ranges recited herein include the endpoints, including those that recite a range “between” two values. The terms “about,” “generally,” “substantially,” and the like are to be construed as modifying a term or value such that it is not an absolute. Such terms will be defined by the circumstances and the terms that they modify as those terms are understood by those of skill in the art. This includes, at very least, the degree of expected experimental error, technique error and instrument error for a given technique used to measure a value.

The content of mono-, di-, and poly-halogenated tyrosine in polypeptides may be determined using methods known in the art. For example, a sample solution is hydrolyzed using acid and/or base. Mono-, di- or poly-halogenated tyrosine standard solutions are prepared by using diluent 1 in Table 4. The standard and sample solutions are analyzed using a LiChroCART® RP18e, or equivalent, column, in an instrument equipped with a UV detector. Additional parameters are as shown in Table 4.

TABLE 4

| | |
|---|---|
| Flow rate | 1.0 mL/minute. |
| Column temperature | 30° C. |
| Wavelength | 220 nm. |
| Diluent | Diluent 1: water. |
| | Diluent 2: 0.1M HCl in water. |
| Buffer | 1.0 mL of orthophosphoric acid in 1 L of Milli Q |
| | water or equivalent. |
| Mobile phase | Mobile phase A: 100% Buffer. |
| | Mobile phase B: Mix buffer and acetonitrile in the |
| | volume ratio 10:90. |
| Injection volume | 10 μL. |
| Run time | 60 minutes. |

The content of mono-, di-, and poly-halogenated tyrosine in a polypeptide sample is determined based on peak areas.

Pharmaceutical compositions comprising a polypeptide, such as glatiramer, of the present application may be formulated using methods known in the art. In embodiments, a liquid composition is lyophilized and subsequently can be dissolved to form an aqueous solution that is suitable for injection. Alternatively, glatiramer acetate may be formulated in any of the forms known in the art for preparing oral, nasal, buccal, and rectal formulations of peptide drugs.

Typically, glatiramer acetate is administered daily to patients suffering from multiple sclerosis, at a dosage of 20 mg.

### DEFINITIONS

The following definitions are used in connection with the present application unless the context indicates otherwise.

The term “polypeptide” as used herein refers to compounds formed from at least two amino acids.

The term “amino acid” as used herein refers to an organic compound comprising at least one amino group and at least one acidic group. The amino acid may be a naturally occurring amino acid or be of synthetic origin, or an amino acid derivative or amino acid analog.

The term “protected amino acids” as used herein, refers to amino acids where functional groups in amino acids are derivatized with any suitable protecting group that can prevent the functional groups from entering into undesired reactions, and can subsequently be readily removed.

The term “protecting group” as used herein, refers to a group attached to functional group of amino acids or peptide or polypeptide that can be cleaved from a peptide or polypeptide under a particular set of conditions. Suitable protecting groups known in the art such as those described in J. F. W. McOmie, “Protective Groups in Organic Chemistry”, Plenum Press, London and New York 1973, in Th. W. Greene, “Protective Groups in Organic Synthesis”, Wiley, New York 1981, in “The peptides”, volume 3 (E. Gross and J. Meienhofer, eds.), Academic Press, London and New York 1981, in “Methoden der organischen Chemie”, Houben-Weyl, 4^{th} edition, Volume 15/I, Georg Thieme Verlag, Stuttgart 1974, in H.-D. Jakubke and H. Jescheit, “Aminosauren, Peptide, Proteine’ (“Amino acids, peptides, proteins”), E. Gross & J. Meienhofer, The Peptides: Analysis, Structure, Biology, Vol. 3: Protection of Functional Groups in Peptide Synthesis (Academic Press, N.Y., 1981); Kricheldorf, H. R. α-Amino Acid N-Carboxy-Anhydride and Related Heterocycles, Springer-Verlag: Berlin, 1987; Blacklock, T. J.; Hirschmann, R.; Veber, D. F. The Peptides; Academic Press: New York, 1987; Vol. 9, p 39.

Certain specific aspects and embodiments will be further explained by the following examples, being provided only for purposes of illustration and not to be construed as limiting the scope of the application in any manner.

### Example 1

### Preparation of Glatiramer Acetate

A N-carboxyanyhydride of L-alanine (1.37 g), a N-carboxyanhydride of L-tyrosine (0.49 g), a N-carboxyanhydride of N-trifluoroacetyl L-lysine, (2.28 g) and a N-carboxyanhydride of γ-benzyl L-glutamate (1.01) are charged into a round bottom flask under a nitrogen atmosphere. 1,4-Dioxane (96 mL) is added at 25-30° C. and the mixture is stirred for 15 minutes. Diethylamine (36 μL) is added at 25-30° C. and the mixture is stirred at the same temperature for 24 hours. The mixture is poured slowly into water (260 mL) and the mass is stirred at 25-30° C. for 30 minutes. The solid is collected by filtration, washed with water (20 mL) and dried under reduced pressure at 28-32° C., to afford 3.86 g of a protected glatiramer.

The protected glatiramer (3.86 g) is charged into a round bottom flask, 33% HBr in acetic acid (38.6 mL) is added, and the mixture is stirred at 25-30° C. for 17 hours. The mixture is slowly added to water (77.2 mL) at 25-30° C. and the mass is stirred for 10 minutes. The solid is collected by filtration, washed with a mixture of water (200 mL) and hexane (50 mL), and dried at 25-30° C. under reduced pressure to afford 2.968 g of trifluoroacetyl glatiramer.

Trifluoroacetyl glatiramer (2.96 g), piperidine (15.9 g), and water (143.6 mL) are charged into a round bottom flask. The mixture is stirred at 25-30° C. for 24 hours and then subjected to diafiltration using a 1 KDa molecular weight cutoff membrane, against ammonium acetate buffer (pH 5.5±0.3), in a stepwise mode of operation, until pH of the permeate reaches 6-6.5. The retentate solution is circulated with 0.3% acetic acid until pH reaches 4.3-4.5 and diafiltered against water to remove excess acetic acid until pH of the retentate reaches 5-5.5. The obtained solution is lyophilized to afford 900 mg of glatiramer acetate.

Peak average molecular weight of glatiramer acetate by GPC: 8403 Daltons; average molar fraction of alanine, glutamic acid, tyrosine and lysine: 0.441, 0.155, 0.080, and 0.323, respectively.

### Example 2

### Preparation of Glatiramer Acetate

A N-carboxyanhydride of L-alanine (5.48 g), a N-carboxyanhydride of L-tyrosine (1.96 g), a N-carboxyanhydride of N-trifluoroacetyl L-lysine (9.12 g) and a N-carboxyanhydride of γ-benzyl L-glutamate (4.04 g) are charged into a round bottom flask under a nitrogen atmosphere. 1,4-Dioxane (384 mL) is added at 25-30° C. and the mixture is stirred for 15 minutes. Diethylamine (144 μL) is added at 25-30° C. and the mixture is stirred at the same temperature for 24 hours under a nitrogen atmosphere. The mixture is poured slowly into water (1000 mL) and the mass is stirred at 25-30° C. for 30 minutes. The solid is collected by filtration, washed with water (80 mL) and dried under reduced pressure at 28-32° C. to afford 15.10 g of a protected glatiramer.

The protected glatiramer (1.0 g) is charged into a round bottom flask. A mixture of concentrated HCl (12 mL) and glacial acetic acid (38 mL) is added and the mixture is stirred at 15-20° C. for 18 hours. The mixture is slowly added to water (250 mL) at 25-30° C. and the mass is stirred for 10 minutes The solid is collected by filtration, washed with a mixture of water (100 mL) and hexane (50 mL) and dried at 25-30° C. under reduced pressure to afford 0.550 g of trifluoroacetyl glatiramer.

Trifluoroacetyl glatiramer (0.40 g), piperidine (2.2 g), and water (19.8 mL) are charged into a round bottom flask. The mixture is stirred at 25-30° C. for 24 hours, then is subjected to diafiltration using a 1 KDa molecular weight cutoff membrane against ammonium acetate buffer (pH 5.5±0.3) in a stepwise mode of operation, until pH of the permeate reaches 6-6.5. The retentate solution is circulated with 0.3% acetic acid until pH reaches 4.3-4.5 and diafiltered against water to remove excess acetic acid, until pH of the retentate reaches 5-5.5. The diafiltered sample is then concentrated through a 3 KDa molecular weight cutoff membrane and the concentrated solution is lyophilized to afford 137 mg of glatiramer acetate.

Peak average molecular weight of glatiramer acetate by GPC: 7662 Daltons.

### Example 3

### Preparation of Glatiramer Acetate

The protected glatiramer from Example 2 (1.0 g) and tetrahydrofuran (200 mL) are charged into a round bottom flask and stirred for 5 minutes at 25-30° C. The mixture is cooled to 0-5° C. and concentrated H₂SO₄ (10 mL) is added at the same temperature. The mixture is stirred at 0-5° C. for 2 hours, then stirred at 25-30° C. for 20 hours. Solvent is distilled from the mixture at 30° C. Water (100 mL) is added to the resulting mass at 25-30° C. and stirred for 10 minutes. The solid is collected by filtration, washed with water (100 mL) and dried at 25-30° C. under reduced pressure to afford 0.510 g of trifluoroacetyl glatiramer.

Trifluoroacetyl glatiramer (0.40 g), piperidine (2.2 g), and water (18 mL) are charged into a round bottom flask. The mixture is stirred at 25-30° C. for 24 hours. The mixture is subjected to diafiltration using a 1 KDa molecular weight cutoff membrane against ammonium acetate buffer (pH 5.5±0.3) in a stepwise mode of operation, until pH of the permeate reaches 6-6.5. The retentate solution is circulated with 0.3% acetic acid until pH reaches 4.3-4.5 and diafiltered against water to remove excess acetic acid until pH of the retentate reaches 5-5.5. The obtained solution is lyophilized to afford 100 mg of glatiramer acetate.

Peak average molecular weight of glatiramer acetate by GPC: 5371 Daltons.

### Example 4

### Preparation of Glatiramer Acetate

A N-carboxyanhydride of L-alanine (5.48 g), a N-carboxyanhydride of L-tyrosine (1.96 g), a N-carboxyanhydride of N-trifluoroacetyl-L-lysine (9.12 g) and a N-carboxyanhydride of γ-benzyl-L-glutamate (4.04 g) are charged into a round bottom flask under a nitrogen atmosphere. 1,4-Dioxane (384 mL) is added at 30° C. and the mixture is stirred for 15 minutes. Diethylamine (144 μL) is added at 25-30° C. and the mixture is stirred at the same temperature for 24 hours under a nitrogen atmosphere. The mixture is poured slowly into water (1000 mL) and the mass is stirred at 25-30° C. for 10 minutes. The solid is collected by filtration, washed with water (20 mL) and dried under reduced pressure at 25-35° C. to afford 15.0 g of a protected glatiramer.

The protected glatiramer (0.5 g) is charged into a round bottom flask. A mixture of 57% of H₁ and H₃PO₂ (5 mL) is added and the mixture is stirred at 30° C. for 17 hours. The mixture is slowly added to water (20 mL) at 30° C. and the mass is stirred for 15 minutes. The solid is collected by filtration, washed with a mixture of water (50 mL) and hexane (20 mL) and dried at 25-30° C. under reduced pressure to afford 0.165 g of trifluoroacetyl glatiramer.

Benzyl chloride content by HPLC: 0.3%.

Trifluoroacetyl glatiramer (110 mg), piperidine (0.6 mL) and water (5.5 mL) are charged into a round bottom flask. The mixture is stirred at 30° C. for 24 hours, then is subjected to diafiltration using a 1 KDa molecular weight cutoff membrane against ammonium acetate buffer (pH 5.5±0.3) in a stepwise mode of operation, until pH of the permeate reaches 6-6.5. The retentate solution is circulated with 0.3% acetic acid until pH reaches 4.3-4.5 and is diafiltered against water to remove excess acetic acid, until pH of the retentate reaches 5-5.5. The diafiltered sample is then concentrated through a 3 KDa molecular weight cutoff membrane and the concentrated solution is lyophilized to afford 68 mg of glatiramer acetate.

Peak average molecular weight of glatiramer acetate by GPC: 4545 Daltons; benzyl chloride content by HPLC: 0.06%.

### Example 5

### Preparation of Glatiramer Acetate

The protected glatiramer from Example 4(A) (1.0 g) is charged into a round bottom flask. A mixture of 57% of H₁ and H₃PO₂ (5 mL) in acetic acid (15 mL) is added and the mixture is stirred at 30° C. for 16 hours. The mixture is slowly added to water (60 mL) at 30° C. and the mass is stirred for 15 minutes. The solid is collected by filtration, washed with a mixture of water (100 mL) and hexane (40 mL), and dried at 25-30° C. under reduced pressure to afford 740 mg of trifluoroacetyl glatiramer.

Benzyl chloride content by HPLC: 0.25%.

Trifluoroacetyl glatiramer (500 mg), piperidine (2.75 mL) and water (25 mL) are charged into a round bottom flask. The mixture is stirred at 30° C. for 24 hours, then is subjected to diafiltration using a 1 KDa molecular weight cutoff membrane against ammonium acetate buffer (pH 5.5±0.3) in a stepwise mode of operation, until pH of the permeate reaches 6-6.5. The retentate solution is circulated with 0.3% acetic acid until pH reaches 4.3-4.5 and diafiltered against water to remove excess acetic acid, until pH of the retentate reaches 5-5.5. The diafiltered sample is then concentrated through a 3 KDa molecular weight cutoff membrane and the concentrated solution is lyophilized to afford 300 mg of glatiramer acetate.

Peak average molecular weight of glatiramer acetate by GPC: 6938 Daltons; benzyl chloride content by HPLC: 0.05%.

### Example 6

### Preparation of Protected Glatiramer

A N-carboxyanhydride of L-alanine (13.56 g), a N-carboxyanhydride of L-tyrosine (4.99 g), a N-carboxyanhydride of N-trifluoroacetyl-L-lysine (22.8 g) and a N-carboxyanhydride of γ-benzyl-L-glutamate (9.89 g) are charged into a round bottom flask under a nitrogen atmosphere. 1,4-Dioxane (996 mL) is added at 25-30° C. and the mixture is stirred for 15 minutes. Diethylamine (360 μL) is added at 25-30° C. and the mixture is stirred at the same temperature for 24 hours. The mixture is poured slowly into water (2.6 L) and the mass is stirred at 25-30° C. for 30 minutes. The solid is collected by filtration, washed with water (1.5 L) and dried under reduced pressure at 25-35° C. to afford 34.5 g of a protected glatiramer.

### Example 7

### Preparation of Glatiramer Acetate

The protected glatiramer from Example-6 (5.0 g) is charged into a round bottom flask at 33° C. with protection from light. A pre-mixed solution of 57% of H₁ and H₃PO₂ (25 mL) in acetic acid (75 mL) is added and the mixture is stirred at 30-35° C. for 17 hours with protection from light. The mixture is slowly added to water (500 mL) at 30-35° C. and the mass is stirred for 15 minutes. The solid is filtered and washed with water (50 mL) to give brown-color compound. The wet compound is washed with 10% sodium thiosulfate solution (Na₂S₂O₃.5H₂O) (5×100 mL) to give white compound, washed with water (2 L) and finally washed with hexane (250 mL) and dried at 25-30° C. under reduced pressure to afford 3.5 g of trifluoroacetyl glatiramer.

Monoiodotyrosine content by HPLC: not detected; diiodotyrosine content by HPLC: not detected.

Trifluoroacetyl glatiramer (3.0 g), piperidine (16.5 mL), and water (150 mL) are charged into a round bottom flask. The mixture is stirred at 25-35° C. for 24 hours, then is subjected to diafiltration using a 1 KDa molecular weight cutoff membrane against ammonium acetate buffer (pH 5.5±0.3) in a stepwise mode of operation, until the pH of the permeate reaches 5.5-6.5. The retentate solution is circulated with 0.3% acetic acid until pH reaches 4.5-4.6 and is diafiltered against water to remove excess acetic acid, until the pH of the retentate reaches 4.8-4.9. The diafiltered sample is then concentrated through a 3 KDa molecular weight cutoff membrane and the concentrated solution is lyophilized to afford 1750 mg of glatiramer acetate.

Peak average molecular weight of glatiramer acetate by GPC: 7988 Daltons; monoiodotyrosine content by HPLC: not detected; diiodotyrosine content by HPLC: not detected.

### Example 8

### Preparation of Glatiramer Acetate

The protected glatiramer from Example 6 (10.0 g) is charged into a round bottom flask at 33° C. with protection from light. A pre-mixed solution of 57% of HI and H₃PO₂ (50 mL) in acetic acid (150 mL) is added and the mixture is stirred at 30-35° C. for 17 hours with protection from light. This reaction mixture is divided in to three equal parts, each of which is further treated separately.

Part 1 of the reaction mixture (180 mL) is charged into water (900 mL) and stirred for 5 minutes. The solid is filtered and washed with water (100 mL) to give a brown-color solid. The wet solid is washed with 10% sodium thiosulfate solution (Na₂S₂O₃.5H₂O) (5×200 mL) to give a white solid, then washed with water (4 L), washed with hexane (500 mL), and dried at 25-30° C. under reduced pressure to afford 6.9 g of trifluoroacetyl glatiramer.

Monoiodotyrosine content by HPLC: not detected; diiodotyrosine content by HPLC: not detected.

Part 2 of the reaction mixture (10 mL) is quenched in 5% ascorbic acid in water (50 mL) and stirred for 5 minutes. The obtained solid is filtered, washed with water (30 mL), washed with hexane (20 mL), and dried at 25-30° C. under reduced pressure to afford 0.15 g of trifluoroacetyl glatiramer.

Monoiodotyrosine content by HPLC: 0.016%; diiodotyrosine content by HPLC: not detected.

Part 3 of the reaction mixture (10 mL) is quenched in water (50 mL) and stirred for 5 minutes. The obtained solid is filtered and washed twice with 5% ascorbic acid in water (50 mL). The resultant solid is washed with water (20 mL), hexane (20 mL) and dried at 25-30° C. under reduced pressure to afford 0.15 g of trifluoroacetyl glatiramer.

Trifluoroacetyl glatiramer of Part 1 (5.0 g), piperidine (27.5 mL) and water (250 mL) are charged into a round bottom flask. The mixture is stirred at 25-35° C. for 24 hours, then is subjected to diafiltration using a 1 KDa molecular weight cutoff membrane against ammonium acetate buffer (pH 5.5±0.3) in a step-wise mode of operation, until the pH of the permeate reaches 5.5-6.5. The retentate solution is circulated with 0.3% acetic acid until the pH reaches 4.5-4.6 and is diafiltered against water to remove excess acetic acid, until the pH of the retentate reaches 4.8-4.9. The diafiltered sample is then concentrated through a 3 KDa molecular weight cutoff membrane and the concentrated solution is lyophilized to afford 3400 mg of glatiramer acetate.

Peak average molecular weight of glatiramer acetate by GPC: 8737 Daltons; monoiodotyrosine content by HPLC: not detected; diiodotyrosine content by HPLC: not detected.

### Example 9

### Preparation of Trifluoroacetyl Glatiramer

Protected glatiramer from Example 6 (2.0 g) is charged into a round bottom flask, 33% HBr in acetic acid (20 mL) is added, and the mixture is stirred at 25-30° C. for 17 hours. The mixture is slowly added to water (40 mL) at 25-30° C. and the mass is stirred for 10 minutes. The solid is filtered, washed with water (100 mL) to give brown-color solid. The wet solid is washed with 10% sodium thiosulfate solution (Na₂S₂O₃.5H₂O) (200 mL) to give white solid, washed with water (200 mL), washed with hexane (100 mL), and dried at 25-30° C. under reduced pressure to afford 1.35 g of trifluoroacetyl glatiramer.

Monoiodotyrosine content by HPLC: 0.36%; Diiodotyrosine content by HPLC: not detected.

### Example 10

### Preparation of Glatiramer Acetate

The protected glatiramer from Example 6 (1.0 g) is charged into a round bottom flask at 30-35° C. with protection from light. A pre-mixed solution of 57% of H₁ and H₃PO₂ (5.0 mL) in acetic acid (15 mL) is added. The mixture is heated to 40° C. and stirred for 4 hours with protection from light. The reaction is quenched with 5% sodium thiosulfate solution (100 mL) and stirred for 10-15 minutes. The solid is filtered, washed with a solution of sodium thiosulfate (50 mL), washed with water (600 mL), washed with hexane (50 mL), and dried at 25-30° C. under reduced pressure, to afford 0.6 g of trifluoroacetyl glatiramer.

Monoiodotyrosine content by HPLC: not detected; Diiodotyrosine content by HPLC: not detected.

Trifluoroacetyl glatiramer (500 mg), piperidine (2.8 mL), and water (25 mL) are charged into a round bottom flask. The mixture is stirred at 25-35° C. for 24 hours, then is subjected to diafiltration using a 1 KDa molecular weight cutoff membrane against ammonium acetate buffer (pH 5.5±0.3) in a stepwise mode of operation, until the pH of the permeate reaches 5.5-6.5. The retentate solution is circulated with 0.3% acetic acid until the pH reaches 4.5-4.6 and is diafiltered against water to remove excess acetic acid, until pH of the retentate reaches 4.8-4.9. The diafiltered sample is then concentrated through a 3 KDa molecular weight cutoff membrane and the concentrated solution is lyophilized to afford 1750 mg of glatiramer acetate.

Monoiodotyrosine content by HPLC: not detected; diiodotyrosine content by HPLC: not detected.

1. A process for preparing a polypeptide or a pharmaceutically acceptable salt thereof, comprising:
(a) polymerizing a mixture of protected amino acids to form a protected polypeptide;
(b) reacting the protected polypeptide with an acid;
(c) optionally, treating the protected polypeptide obtained in step (b) with a reagent to reduce the content of molecular impurities; and
(d) reacting the protected polypeptide obtained in steps (b) or (c) with a base to form a polypeptide or a pharmaceutically acceptable salt thereof.

2. The process of claim 1, wherein the amino acids are L-tyrosine, L-alanine, L-glutamate, and L-lysine.

3. The process of claim 1, wherein the polypeptide is glatiramer.

4. The process of claim 1, wherein protected amino acids are amino acid N-carboxyanhydrides.

5. The process of claim 1, wherein an acid comprises one or more of acetic acid, propionic acid, butyric acid, hydrochloric acid, hydrogen bromide, hydrogen fluoride, hydrogen iodide, methanesulfonic acid, trifluoromethanesulfonic acid, phosphorous acid, trifluoroacetic acid, sulfuric acid, phosphoric acid, and hypophosphoric acid.

6. The process of claim 1, wherein an acid comprises two or more of acetic acid, propionic acid, butyric acid, hydrogen chloride, hydrogen bromide, hydrogen fluoride, hydrogen iodide, methanesulfonic acid, trifluoromethanesulfonic acid, phosphorous acid, trifluoroacetic acid, sulfuric acid, phosphoric acid, and hypophosphoric acid.

7. The process of claim 1, wherein an acid comprises two or more of acetic acid, hydrogen chloride, hydrogen bromide, hydrogen iodide, and hypophosphoric acid.

8. The process of claim 1, wherein an acid comprises at least one of hydrogen chloride, hydrogen bromide, hydrogen iodide, sulfuric acid, and hypophosphoric acid.

9. The process of claim 1, wherein a reagent comprises one or more of sodium thiosulfate, sodium bisulfate, sodium metabisulfite, ascorbic acid, activated carbon fiber, an ion exchange resin, a silver salt, and sodium bicarbonate.

10. The process of claim 1, wherein a base is an organic base.

11. The process of claim 1, wherein a base is an inorganic base.

12. The process of claim 1, wherein a base comprises piperidine.

13. A process for preparing glatiramer or a pharmaceutically acceptable salt thereof, comprising:
(a) polymerizing a mixture of the protected amino acids L-tyrosine, L-alanine, L-glutamate, and L-lysine to form protected glatiramer;
(b) reacting the protected glatiramer with an acid;
(c) optionally, treating the protected polypeptide obtained in step (b) with a reagent to reduce the content of molecular impurities; and
(d) reacting the protected polypeptide obtained in steps (b) or (c) with a base to form a polypeptide or a pharmaceutically acceptable salt thereof.

14. The process of claim 13, wherein the protected amino acids are amino acid N-carboxyanhydrides.

15. The process of claim 13, wherein an acid comprises one or more of acetic acid, propionic acid, butyric acid, hydrochloric acid, hydrogen bromide, hydrogen fluoride, hydrogen iodide, methanesulfonic acid, trifluoromethanesulfonic acid, phosphorous acid, trifluoroacetic acid, sulfuric acid, phosphoric acid, and hypophosphoric acid.

16. The process of claim 13, wherein an acid comprises two or more of acetic acid, propionic acid, butyric acid, hydrogen chloride, hydrogen bromide, hydrogen fluoride, hydrogen iodide, methanesulfonic acid, trifluoromethanesulfonic acid, phosphorous acid, trifluoroacetic acid, sulfuric acid, phosphoric acid, and hypophosphoric acid.

17. The process of claim 13, wherein an acid comprises two or more of acetic acid, hydrogen chloride, hydrogen bromide, hydrogen iodide, and hypophosphoric acid.

18. The process of claim 13, wherein an acid comprises at least one of hydrogen chloride, hydrogen bromide, hydrogen iodide, sulfuric acid, and hypophosphoric acid.

19. The process of claim 13, wherein a reagent comprises one or more of sodium thiosulfate, sodium bisulfate, sodium metabisulfite, ascorbic acid, activated carbon fiber, an ion exchange resin, a silver salt, and sodium bicarbonate.

20. The process of claim 13, wherein a base is an organic base.

21. The process of claim 13, wherein a base is an inorganic base.

22. The process of claim 13, wherein a base comprises piperidine.

23. A process for preparing a polypeptide or a pharmaceutically acceptable salt thereof, comprising:
(a) polymerizing a mixture of protected amino acids to form a protected polypeptide; and
(b) reacting the protected polypeptide with an acid to form a polypeptide or a pharmaceutically acceptable salt thereof.

24. The process of claim 23, wherein protected amino acids are amino acid N-carboxyanhydrides.

25. The process of claim 23, wherein the amino acids are L-tyrosine, L-alanine, L-glutamate, and L-lysine.

26. The process of claim 23, wherein the polypeptide is glatiramer.

27. The process of claim 23, wherein an acid comprises one or more of acetic acid, hydrogen iodide, phosphorous acid, phosphoric acid, and hypophosphoric acid.

28. The process of claim 23, wherein an acid comprises two or more of acetic acid, phosphorous acid, phosphoric acid, and hypophosphoric acid.

29. The process of claim 23, wherein an acid comprises two or more of acetic acid, hydrogen iodide, and hypophosphoric acid.

30. The process of claim 23, wherein an acid comprises hydrogen iodide.

31. The process of claim 13, further comprising purifying a polypeptide or a pharmaceutically acceptable salt thereof.

32. The process of claim 13, further comprising purifying glatiramer or a pharmaceutically acceptable salt thereof.

33. The process of claim 23, further comprising purifying a polypeptide or a pharmaceutically acceptable salt thereof.

34. The process of claim 23, further comprising purifying glatiramer or a pharmaceutically acceptable salt thereof.
